(19) United States
(12) Patent Application Publication
(10) Pub. No.: US 2023/0062256 A1
(43) Pub. Date: Mar. 2, 2023

(54) MULTI-MODAL IMAGING FOR CELL TRACKING

(71) Applicant: THE REGENTS OF THE UNIVERSITY OF MICHIGAN, Ann Arbor, MI (US)

(72) Inventors: Xudong FAN, Ann Arbor, MI (US); Xueding WANG, Ann Arbor, MI (US); Xuzhou LI, Ann Arbor, MI (US); Wei ZHANG, Ann Arbor, MI (US); William Yanli WANG, Ann Arbor, MI (US); Xiaoqin WU, Ann Arbor, MI (US); Xiaotian TAN, Ann Arbor, MI (US); Brendon BAKER, Ann Arbor, MI (US); Yannis M. PAULUS, Ann Arbor, MI (US)

(73) Assignee: THE REGENTS OF THE UNIVERSITY OF MICHIGAN, Ann Arbor, MI (US)

(21) Appl. No.: 17/795,581

(22) PCT Filed: Jan. 28, 2021
(86) PCT No.: PCT/US2021/015475

§ 371 Date: Jul. 27, 2022

Related U.S. Application Data
(60) Provisional application No. 62/967,209, filed on Jan. 29, 2020.

(51) Int. Cl. A61B 3/12; G01N 33/58; A61B 3/10; G01N 21/47; G01N 21/64

(52) U.S. Cl. CPC A61B 3/1241; A61B 3/102; A61B 3/1233; G01N 21/4795; G01N 21/6428; G01N 33/582; A61B 8/10

(57) ABSTRACT

An imaging system for cell-based therapies is provided. The imagining system includes one or more optical tags configured for insertion into a cell or biological tissue, an excitation light source configured to illuminate the one or more optical tags; a detector configured to measure optical emission of the one or more optical tags; an imaging subsystem configured to determine a three-dimensional location of each of the one or more optical tags in the cell or biological tissue; and a controller in electrical communication with the excitation light source, the detector, and the imaging subsystem. Each of the one or more optical tags has a contrasting feature and includes a fluorescent material. The contrasting feature may be defined by at least one of a refractive index, shape, color, and laser emission of each optical tag of the one or more optical tags.

### CROSS-REFERENCE TO RELATED APPLICATION

This application claims the benefit of U.S. Provisional Application No. 62/967,209, filed on Jan. 29, 2020. The entire disclosure of the above application is incorporated herein by reference.

### GOVERNMENT CLAUSE

This invention was made with government support under ECCS1607250 awarded by the National Science Foundation. The government has certain rights in the invention.

### FIELD

The present disclosure relates to multi-modal imaging for cell tracking.

### BACKGROUND

Cell-based therapies, such as stem-cell therapy and immunotherapy, can be used as therapeutics in biological research and clinical practices. In vivo cell distribution, migration, and development are commonly used to evaluate the efficiency of such therapeutic treatments. Current assessments often include indirect methods, such as evaluating the efficiency of treatment by the size change or recovery of a target area (e.g., tumor). Direct methods are important for both the application of cell-based therapies and for the exploration of mechanisms in basic biological research.

Optical coherence tomography (“OCT”) is a clinically applicable imaging technology with ultrahigh-resolution and excellent imaging depth. It also demonstrates potential for in-vivo cellular imaging studies, especially for cell dynamic researches. However, due to the slight scattering difference between cells and surrounding tissue layers, original OCT imaging contrasts of cell dynamics are often poor. Therefore, contrast agents, such as gold nanoparticles and polymer microspheres with strong scattering properties uptaken by targeted cells are often used to improve the visibility and acquire better images. However, due to the homogeneity of the cell labeling contrast agents used in current OCT systems, only the change of the overall distribution of the entire cell population can be observed and each individual cell cannot be independently tracked. Since fluorescence labeling is a widely used technique to differentiate targets for cell migrations, fluorescence labels have also been used along with the intensity-based grayscale OCT images to provide distinguishable information to track different cell groups. However, as a result of the broad emission spectra of fluorophores, the number of different labels is limited by its nature so that the technique can only be used to label groups of cells and not each individual cell.

The use of laser emission-based imaging systems and methods in biomedical research has recently emerged. For example, microlasers have shown promise as intracellular labels in part due to their small sizes, high emission intensities, rich emission spectra, and narrow linewidths. Since the size of microlasers is small, microlasers can be internalized by cells and laser emissions are able to detect microenvironment changes. Because the lasing emission spectra are primarily determined by distinct microlaser resonant cavities and structures, various lasing emission spectra data can be acquired and used as unique “identifiers” to label each individual cell. Microfabricated whispering gallery mode (“WGM”) lasers have been used to provide a “barcode” type of identifications for tracking a massive number of cells in-vivo. The application of microlaser cell tracking, however, is hindered for in-vivo studies at least because the lasing emission of microlasers may be blurred because of the omnipresent scattering tissue such that the tracking position is not precise, and because lasing-emission-based cell tracking technology itself is only able to provide two-dimensional (“2D”) images. Three-dimensional (“3D”) information is critical for locating spatial positions during in-vivo studies. Accordingly, there is a need for systems and methods that are able to more easily and accurately track individual in-vivo cell migration in three-dimensions.

This section provides background information related to the present disclosure which is not necessarily prior art.

### SUMMARY

This section provides a general summary of the disclosure, and is not a comprehensive disclosure of its full scope of all of its features.

In various aspects, the present disclosure provides an imaging system for cell-based therapies. The imagining system may include one or more optical tags configured for insertion into a cell or biological tissue, where each of the one or more optical tags has a contrasting feature and includes a fluorescent material; an excitation light source configured to illuminate the one or more optical tags; a detector configured to measure optical emission of the one or more optical tags; an imaging subsystem configured to determine a three-dimensional location of each of the one or more optical tags in the cell or biological tissue; and a controller in electrical communication with the excitation light source, the detector, and the imaging subsystem.

In one aspect, the imaging subsystem may be configured to determine the three-dimensional location of the one or more optical tags using optical coherence tomography (“OCT”).

In one aspect, each of the one or more optical tags may exhibit a first refractive index that is different from a second refractive index of a surrounding medium of the cell or biological tissue. The first refractive index may define the contrasting feature.

In one aspect, the one or more optical tags includes a first optical tag and a second optical tag. The first optical tag may exhibit a first refractive index. The second optical tag may exhibit a second refractive index. At least one of the first and second refractive indexes may be different from a third refractive index exhibited by a surrounding medium of the cell or biological tissue or the first refractive index may be different from the second refractive index. The first, second, and third refractive indexes may define the contrasting feature.

In one aspect, the one or more optical tags may include a first optical tag and a second optical tag. The first optical tag may have a first shape. The second optical tag may have a second shape that is different from the first shape. The first and second shapes may define the contrasting feature.

In one aspect, the one or more optical tags may include a first optical tag and a second optical tag. The first optical tag may have a first laser emission. The second optical tag may have a second laser emission that is different from the first laser emission. The first and second laser emissions may define the contrasting feature.

In one aspect, the one or more optical tags may include a first optical tag and a second optical tag. The first optical tag may have a first color. The second optical tag may have a second color that is different from the first color. The first and second colors may define the contrasting feature.

In one aspect, at least one optical tag of the one or more optical tags has a core-shell structure including an air-containing core and a polymeric shell. The air-containing core and the polymeric shell may define the contrasting feature.

In one aspect, the imaging subsystem may be configured to determine the three-dimensional location of the one or more optical tags using an ultrasound imagining subsystem.

In one aspect, the one or more optical tags includes a first optical tag and a second optical tag. The first optical tag may exhibit a first refractive index. The first refractive index may be different from a second refractive index of a surrounding medium of the cell or biological tissue. The second optical tag may have a core-shell structure including an air-containing core and a polymeric shell. The first refractive index, the air-containing core, and the polymeric shell may define the contrasting feature.

In one aspect, the one or more optical tags may be independently selected from a group consisting of: nanowires, semiconductor ring resonators, microspheres, photonic crystals, and combinations thereof.

In one aspect, at least one optical tag of the one or more optical tags may be coated with a biocompatible coating.

In one aspect, the biocompatible coating may include one or more materials selected from the group consisting of: diallyl dimethylammonium chloride (PDDA), polyacrylic acid (PAA), collagen, polyethylene glycol (PEG), polydimethylsiloxane (PDMA), and combinations thereof.

In one aspect, the biocompatible coating may include a first coating and a second coating. The first coating may surround the at least one optical tag of the one or more optical tags. The second coating may surround the first coating. The first and second coatings may each include one or more materials independently selected from the group consisting of: diallyl dimethylammonium chloride (PDDA), polyacrylic acid (PAA), collagen, polyethylene glycol (PEG), polydimethylsiloxane (PDMA), and combinations thereof.

In one aspect, each of the one or more optical tags may have a largest dimension less than about 20 µm.

In one aspect, each of the one or more optical tags may have an length greater than or equal to about 3 µm to less than or equal to about 10 µm, and an average diameter of about 200 nm.

In one aspect, the excitation light source may be a pulsed laser.

In one aspect, the detector may be at least one of a photodiode or a spectrometer.

In various aspects, the present disclosure provides an imaging system for cell-based therapies. The imagining system may include one or more optical tags configured for insertion into a cell or biological tissue, where each of the one or more optical tags has a contrasting feature and includes a fluorescent material; an excitation light source configured to illuminate the one or more optical tags; a detector configured to measure optical emission of the one or more optical tags; an optical coherence tomography (“OCT”) imaging subsystem configured to determine a three-dimensional location of each of the one or more optical tags in the cell or biological tissue; and a controller in electrical communication with the excitation light source, the detector, and the imaging subsystem. The contrasting features of each of the one or more optical tags may be defined by at least one of a refractive index, shape, color, and laser emission.

In one aspect, at least one optical tag of the one or more optical tags has a core-shell structure including an air-containing core and a polymeric shell. The air-containing core and the polymeric shell may define another contrasting feature.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic illustration of an example multi-modal imaging system in accordance with various aspects of the present disclosure.

FIG. 2 is a schematic illustration of another example multi-modal imaging system in accordance with various aspects of the present disclosure.

FIG. 3 is a schematic illustration of an example configuration for the multi-modal imaging system illustrated in FIG. 1.

FIG. 4 is a schematic illustration of an example optical tag having a biocompatible coating in accordance with various aspects of the present disclosure.

FIG. 5A is a confocal fluorescence image of example optical tags internalized by target cells in accordance with various aspects of the present disclosure, where the upper inset is an enlarged top view of an example target cell and the lower inset is an enlarged side view of the example target cell.

FIG. 5B is a lasing spectrum of an example optical tag internalized by target cells in accordance with various aspects of the present disclosure, where the inset is the threshold curve with a 50 µm scale bar.

FIG. 6A is a fluorescence image of target cells labeled using optical tags and cultured on a hydrogen gel layer in accordance with various aspects of the present disclosure, where four target cells are specifically identified.

FIG. 6B is a two-dimensional X-Y plane optical coherence tomography image of the same field of view illustrated in FIG. 6A.

FIG. 6C are lasing spectra collected from the four tracked cells as shown in FIGS. 6A and 6B, with a 100 µm scale bar.

FIG. 7A is a fluorescence image tracking target cells labeled using optical tags and cultured on a hydrogen gel layer in accordance with various aspects of the present disclosure, where the right inset is a demonstration of an example target cell over time through multiple frames.

FIG. 7B are normalized lasing spectra collected for target cell migration tracking over time in accordance with various aspects of the present disclosure.

FIG. 7C show the in-vivo three-dimensional target cell migration trajectories extracted from multiple optical coherence tomography scans over 24 hours in accordance with various aspects of the present disclosure.

FIG. 8A is a color fundus image of an albino rabbit injected with one or more optical tags in accordance with various aspects of the present disclosure.

FIG. 8B is an optical coherence tomography B-scan image collected two days after the sub-retinal injection illustrated in FIG. 8A, where the right inset is a signal intensity curve collected along the dashed line.

FIG. 8C is a side view of the three-dimensional optical coherence tomography retinal layer reconstruction and the distribution of the optical tags after the sub-retinal injection illustrated in FIG. 8A.

FIG. 8D is a top view of the three-dimensional optical coherence tomography retinal layer reconstruction and the distribution of the optical tags in the same field of view as FIG. 8C.

FIG. 8E is a two-dimensional X-Y plane optical coherence tomography image of the retinal layer reconstruction and the distribution of the optical tags in the same field of view as FIG. 8C.

FIG. 8F is a fluorescence image of the retinal layer reconstruction and the distribution of the optical tags in the same field of view as FIG. 8C.

FIG. 8G is the unique lasing spectra collected from the optical tags identified in FIGS. 8E and 8F.

FIG. 9A illustrates the in-vivo fluorescence two-dimensional optical tag migration through multiple observations, where three target cells are specifically identified, in accordance with various aspects of the present disclosure.

FIG. 9B depicts a normalized lasing spectra collected for migration of the optical tags illustrated in FIG. 9A over time.

FIG. 9C illustrates the corresponding in-vivo three-dimensional migration trajectory of the optical tags illustrated in FIGS. 9A and 9B extracted from multiple optical coherence tomography scans over three days.

Corresponding reference numerals indicate corresponding parts throughout the several views of the drawings.

### DETAILED DESCRIPTION

Example embodiments will now be described more fully with reference to the accompanying drawings.

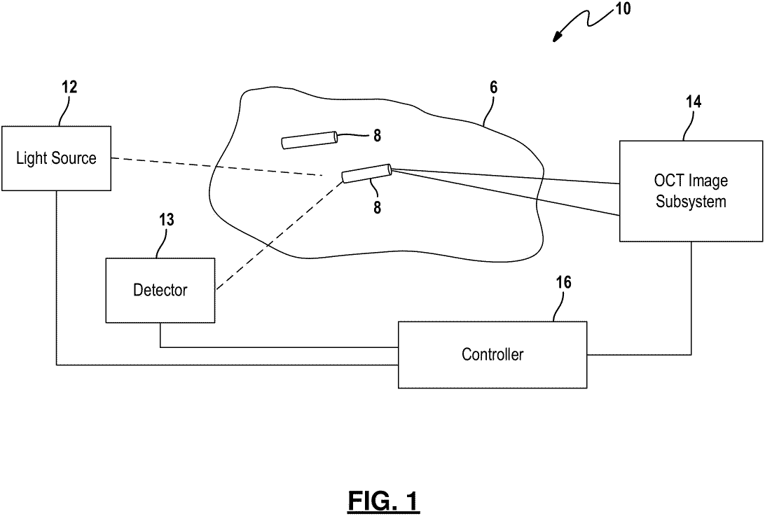

Systems for tracking individual in-vivo cell migration in three dimensions are provided. For example, FIG. 1 illustrates a multi-modal imaging system 10 configured to identify and track individual cells (e.g., macrophages). The imaging system 10 is configured to detect and measure the position of one or more optical tag 8 inserted into a biological tissue 6. In at least some example embodiments, the one or more optical tags 8 may each have two or more purposes or functions or modalities. For example, each optical tag 8 may be labeled or doped with a fluorescent material, such as a dye, quantum dots, rear earth metals, and the like. As further discussed below, in this manner, the one or more optical tags 8 may serve as optical spectral tags, in which the fluorescence or laser emission intensity and spectral can be used to categorize individual or different cells in the target tissue 6. Each optical tag 8 may also have different refractive indices and/or different geometric sizes and shapes such that the one or more optical tags 8 may serve as a contrast agent for optical coherence tomography (“OCT”) imaging. For example, each of the one or more optical tags 8 may exhibit a refractive index that is higher than a refractive index of the target tissue 6.

For tracking multiple cells, different optical tags 8 may be embedded into different cells (not shown) of the target tissue 6. For example, optical tags 8 with different fluorescent markers and/or refractive indices may be used to identity different target cells 6. In at least some example embodiments, optical tags 8 with different geometric shapes and/or sizes can also be used to identify different cells. In this way, the optical tags 8 provide a set of identifiers to categorize individual and different cells. Cells can also be identified using a combination of these attributes. The total number of identifiers can be calculated by multiplying the number of unique fluorescent attributes by the number of unique OCT attributes. For example, assuming that each of the one or more optical tags 8 has two levels of refractive indices, two levels of sizes, and one-hundred different fluorescent features, four-hundred different identifiers can be generated to track up to four-hundred different cells.

In some example embodiments, the one or more optical tags 8 are CdS nanowires, which may have two facts that work as two mirrors. In other example embodiments, the one or more optical tags 8 may be semiconductor ring resonators, such as silicon ring resonators doped with fluorescent or photoluminescent materials, polymer or glass microspheres doped with dyes or rare earth materials, high-index microspheres doped with dyes or rare earth materials, and/or photonic crystal doped with dyes or other fluorescent or photoluminescent materials, each material may have mirror-like features. These examples are intended to be illustrative and not limiting of different implementations for the one or more optical tags 8.

Further, though not illustrated, the skilled artisan will appreciate that in at least one example embodiment, in addition to the one or more optical tags 8, one or more functional probes, such as molecular beacons, may be also inserted into the target cell and/or biological tissue 6. The one or more functional probes may be configured to detect various cell activity.

In each instances, the largest dimension of each of the one or more optical tags 8 is preferably less than about 1 mm, and in certain aspects, optionally less than about 20 µm. For example, in at least one example embodiment, the one or more optical tags 8 may have a length greater than or equal to about 3 µm to less than or equal to about 10 µm, and an average diameter of about 200 nm.

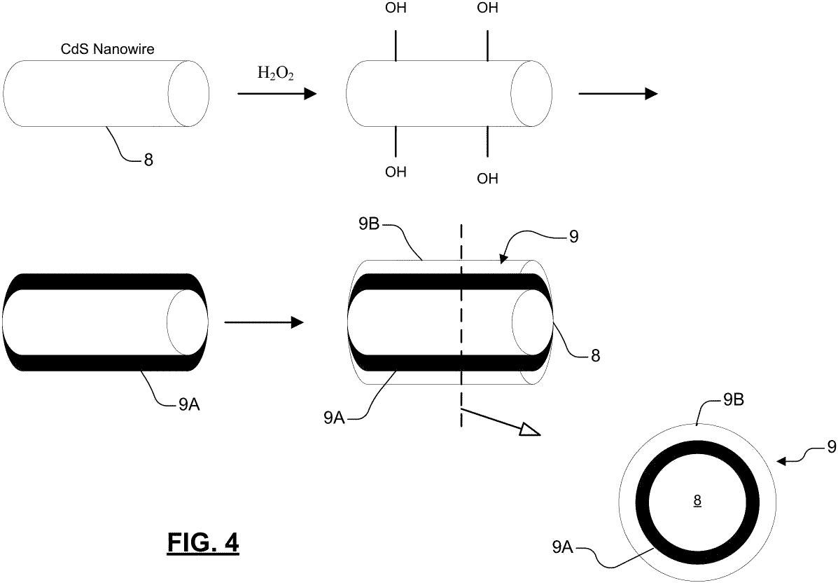

In at least some example embodiments, as illustrated in FIG. 4, each of the one or more optical tags 8 may be coated with a biocompatible coating 9. The biocompatible coating 9 may extend continuously so as to substantially surround the optical tag 8. The biocompatible coating 9 may have a thickness greater than or equal to about 1 nm to less than or equal to about 2000 nm and may include one or more materials selected from the group consisting of: diallyl dimethylammonium chloride (PDDA), polyacrylic acid (PAA), collagen, polyethylene glycol (PEG), polydimethylsiloxane (PDMA), and combinations thereof. In at least one example embodiment, the biocompatible coating 9 includes a first biocompatible coating 9A and a second biocompatible coating 9B. As illustrated, the first biocompatible coating 9A may be disposed on or adjacent to the optical tag 8, and the second biocompatible coating 9B may be disposed on or adjacent to an exposed surface of the first biocompatible coating 9B. The first and second biocompatible coatings 9A, 9B may each include one or more materials independently selected from the group consisting of: diallyl dimethylammonium chloride (PDDA), polyacrylic acid (PAA), collagen, polyethylene glycol (PEG), polydimethylsiloxane (PDMA), and combinations thereof. For example, in at least one example embodiment, the first biocompatible coating 9A may include diallyl dimethylammonium chloride (PDDA) and polyacrylic acid (PAA), and the second biocompatible coating 9B may comprise collagen.

In at least some example embodiments, the one or more optical tags 8 may be synthesized using a gold-nanocluster catalyzed vapor-liquid-solid method. For example, CdS nanowires may be prepared by placing CdS powders (e.g., Sigma-Aldrich, 99.99% purity) on an alumina boat in the center of a heating zone inside a horizontal quartz tube mounted in a single zone furnace. Silicon wafers (e.g., QI Electronics Inc.) covered with a 10 nm-thick gold film (for example, by sputtering) may be located downstream from CdS powders near the end of the heating zone. A high purity nitrogen gas flow with a flow rate of about 700 SCCM may be introduced into the system to purge oxygen out. After about a one hour gas flow cleaning, the furnace may be heated from room temperature to about 850° C. at about 500 mbar pressure and kept for another extra hour. Meanwhile, a 155 SCCM nitrogen gas flow may be maintained in the whole heating process so as to transport the evaporated CdS vapor to the gold-catalyzed silicon substrates to initialize nanowire growth. After growing for one hour, yellowish nanowire products can be found on the silicon substrate.

With renewed reference to FIG. 1, in at least some example embodiments, the imaging system 10 includes an excitation light source 12 and a detector 13. For the fluorescent modality, the excitation light source 12 may be configured to illuminate the one or more optical tags 8 placed in the target 6, and the detector 13 may be configured to measure fluorescent emission from the one or more optical tags 8. In at least some example embodiments, the excitation light source 12 is a pulsed laser, although other types of light sources are also contemplated by this disclosure. In at least some example embodiments, the detector 12 is an avalanche photodiode, a spectrometer, or both an avalanche photodiode and a spectrometer, although other types of detectors are also contemplated by this disclosure.

The imaging system 10 also includes an imaging subsystem 14. For the OCT modality, the imaging subsystem 14 is configured to determine the three-dimensional location of the one or more optical tags 8 using optical coherence tomography (“OCT”).

In at least some example embodiments, the imaging system 10 also includes a controller 16. The controller 16 is in electrical communication with the excitation light source 12, the detector 13, and the imaging subsystem 14. The controller 16 may be a microcontroller. It should be understood that the logic for the control of imaging system by controller 16 can be implemented in hardware logic, software logic, or a combination of hardware and software logic. In this regard, the controller 16 can be or can include any of a digital signal processor (DSP), microprocessor, microcontroller, or other programmable device, which are programmed with software for implementing the described methods. It should be understood that alternatively the controller 16 is or includes other logic devices, such as a Field Programmable Gate Array (FPGA), a complex programmable logic device (CPLD), or application specific integrated circuit (ASIC). When it is stated that controller 16 performs a function or is configured to perform a function, it should be understood that controller 16 is configured to do so with appropriate logic (such as in software, logic devices, or a combination thereof).

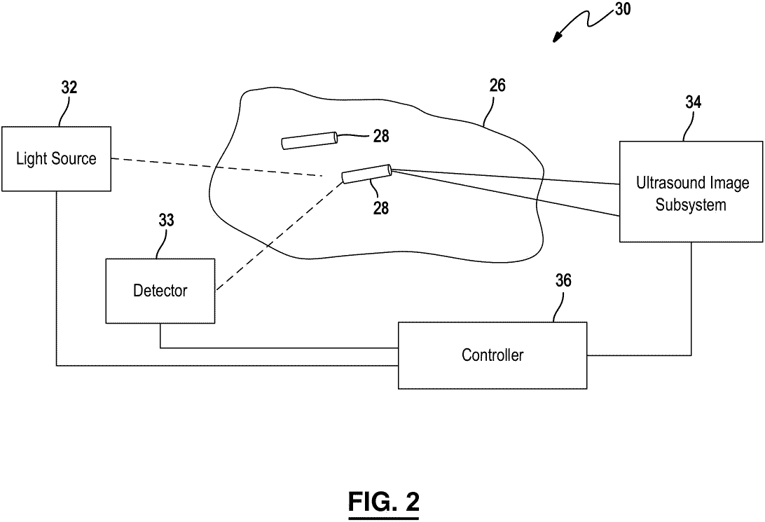

FIG. 2 illustrates a multi-modal imaging system 30 configured to identify and track individual cells. Like imaging system 10, imaging system 30 is configured to detect and measure the position of one or more optical tags 28 inserted into a biological tissue 26. In at least some example embodiments, the one or more optical tags 28 may each have two or more purposes or functions. For example, each optical tag 28 may be labeled or doped with a fluorescent material such that the one or more optical tags 28 may serve as optical spectral tags. Each optical tag 28 may also serve as an acoustic contrast agent. For example, though not illustrated, each of the one or more optical tags 28 may be a hollow or core/shell-structured microsphere include an air-containing core and a polymeric shell that may be doped with a dye, which can be a type of fluorescent material. During operation, the air inside the microsphere may be used as the acoustic contrast agent while the dye in the shell may be used for optical tracking.

Like imaging system 10, in at least some example embodiments, the imaging system 30 includes an excitation light source 32, a detector 33, an imaging subsystem 34, and a controller 36. Like controller 16, controller 36 is in electrical communication with the excitation light source 32, the detector 33, and the imaging subsystem 34. For the fluorescent modality, the excitation light source 32 may be configured to illuminate the one or more optical tags 28 placed in the target 26, and the detector 33 may be configured to measure fluorescent emission from the one or more optical tags 28. The imaging subsystem 34 may be an ultrasound imagining subsystem configured to determine the three-dimensional location of the one or more optical tags 28.

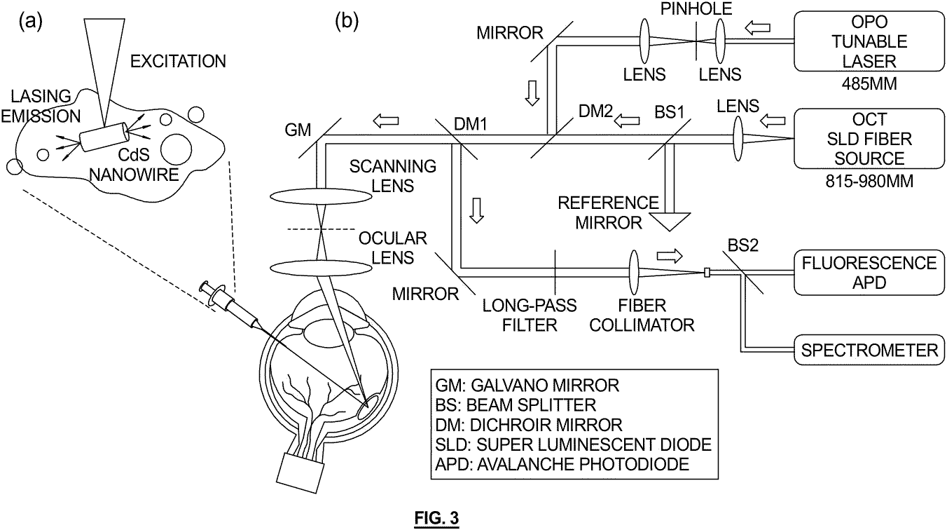

FIG. 3 is a schematic illustration of an example configuration for an example multi-modal imaging system having integrated spectral-domain optical coherence tomography (“SD-OCT”) and fluorescence microscopy (FM) with laser emission, like the multi-modal imaging system 10 illustrated in FIG. 1. For example, a commercially available SD-OCT system (such as available from Thorlabs (Ganymede-II-HR, Thorlabs)) may be modified to include an ocular lens (OL) between the target and a scan lens. For FM and laser excitation, an optical parametric oscillator (OPO) (such as, NT-242, Ekspla, tunable wavelength ranging from about 405 nm to about 2600 nm, pulse duration ranging from about 3 ns to about 6 ns) can be used as an illumination source. The fluorescence and laser emission may be collected from the backward optical path of telescope configuration, reflected by a dichroic mirror (“DM”) and separated by a fiber optic coupler (such as, TN532R2F1, Thorlabs) after passing through the long-pass filter (such as, FGL495M, Thorlabs) into fluorescence and laser emission channels. The fluorescence signal may be detected by an avalanche photodiode (APD) (such as, APD 130A, Thorlabs,) and digitized by a DAQ card (such as, PX1500-4, Signatec Inc, sampling rate 500 MHz). The laser emission spectra were collected by the spectrometer with an integration time of 0.5 s (Ocean Optics, HR4000). The OPO laser, the OCT scan header, and the data acquisition (“DAQ”) card were synchronized through a multifunction I/O device (such as, USB-6353, National Instruments Corporation). A delay generator and a clock switch circuit may be applied to switch the system clock between the OPO and OCT systems. The light from different imaging modalities may be coaxially aligned to ensure the co-registration of the multimodality images. In at least some example embodiments, the lateral and axial resolution of SD-OCT may be about 3.8 µm and about 4.0 µm respectively. Further, a laser wavelength of about 485 nm and a laser energy of about 150 nJ before the eye may be used.

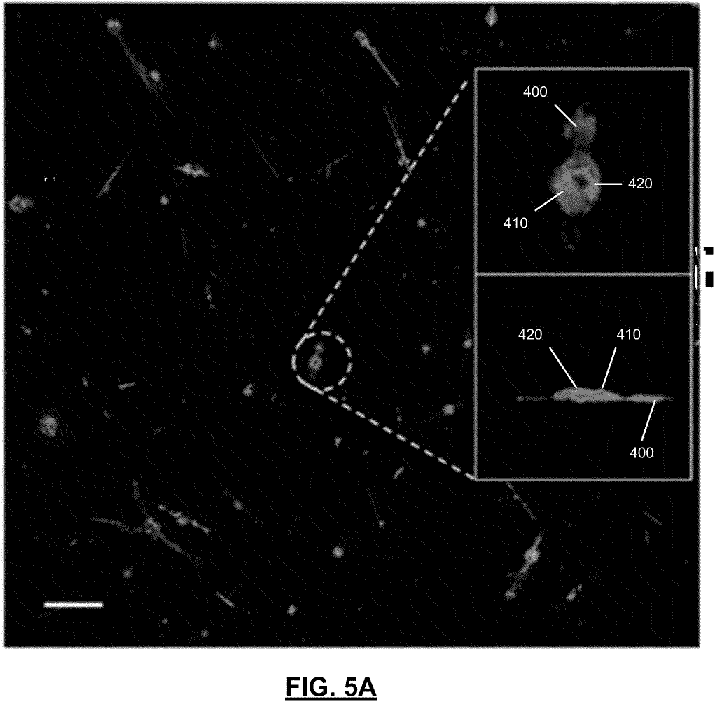

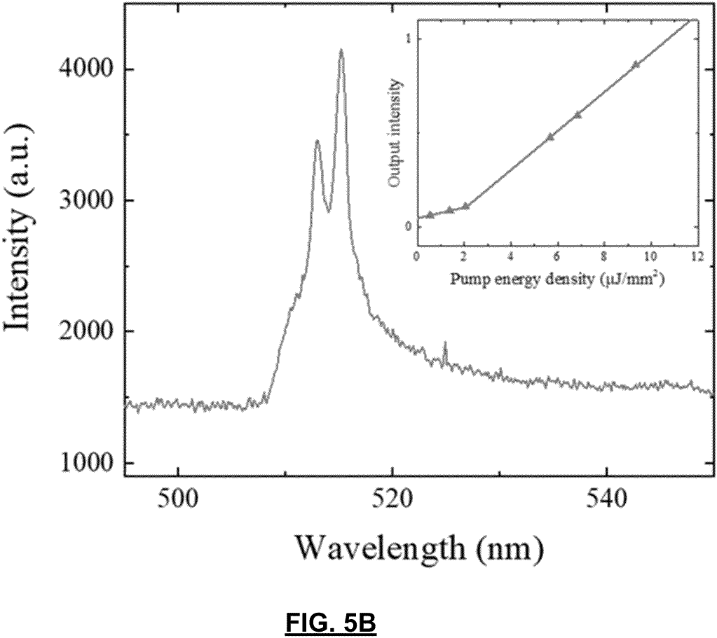

FIG. 5A is a confocal fluorescence image (such as can be obtained using a Zeiss LSM800 confocal microscope) showing internalization and characterization of dual-functional optical tags, such as optical tags 8 illustrated in FIG. 1 and/or optical tags 28 illustrated in FIG. 2, in target cells (e.g., macrophages). The cell cytoskeletons were stained blue 400 and the nuclei were stained red 410. The optical tags can be visualized by their own green fluorescence 420 under 488 nm laser excitation. From the enlarged cross-section images, the upper inset showing an enlarged top view of an example target cell and the lower inset showing an enlarged side view of the example target cell, the full internalization of optical tags in the target cells is verified.

FIG. 5B is a lasing spectrum of dual-functional optical tags, such as optical tags 8 illustrated in FIG. 1 and/or optical tags 28 illustrated in FIG. 2. Due to the high refractive index (e.g., n = 2.67), the reflection on each optical tag end-facet may be high, which could perform as two reflective mirrors. Therefore, the two endfacets of each optical tag may build an analogous “Fabry-Perot” (F-P) cavity with a quality factor (“Q factor”) of about 50, where the optical tags serve as the gain media. Even though the Q-factor is relatively low, optical tags can provide a considerably high gain up to about 3000/cm at a carrier density of 1-3 × 10⁻¹⁹ /cm³ so as to compensate for cavity loss and to generate lasing emission. An exemplary lasing emission spectrum of an example dual-functional optical tag (e.g., CdS nanowire) internalized by a target cell (e.g., macrophage) is illustrated in FIG. 5B. The inset is the lasing output versus pump energy curve and illustrates a lasing threshold of about 1 µJ/mm². In addition, due to the high refractive index (e.g., about 2.67) of the optical tags and the elongated shape, the optical tags have strong scattering properties. As such, the optical tags may generate significant signal enhancement when introduced into the OCT imaging. Along with its own fluorescence emission, the optical tags have exceptional potential to be employed as an OCT-fluorescence dual-modality imaging contrast agent.

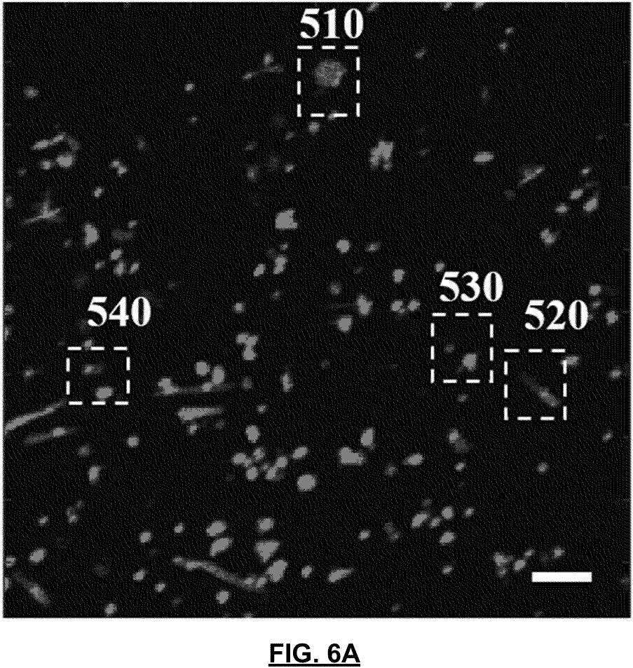

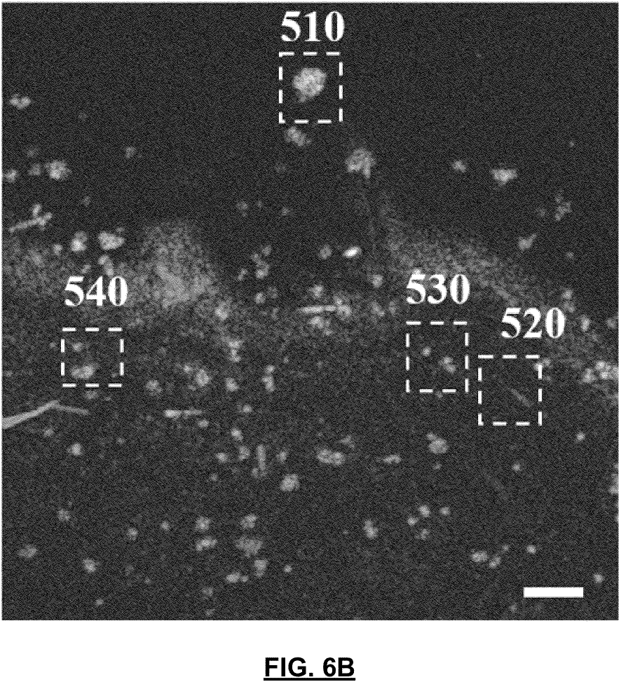

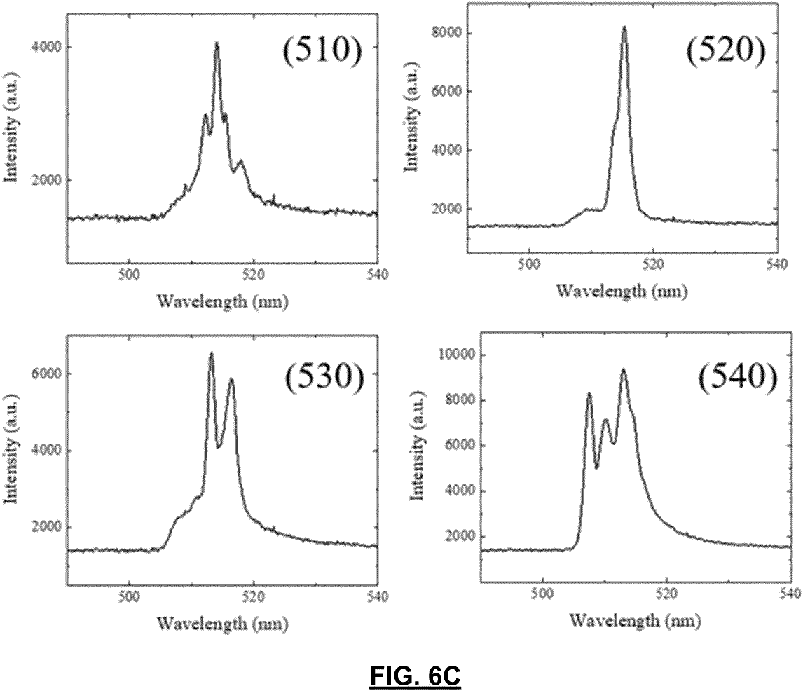

FIG. 6A is a fluorescence image of target cells (e.g., macrophages) labeled using optical tags (e.g., CdS nanowires) and cultured on a layer of scattering hydrogen gel; and FIG. 6B is a two-dimensional X-Y plane optical coherence tomography image of the same field of view illustrated in FIG. 6A. FIGS. 6A and 6B together show in vivo tracking abilities of the dual-functional optical tags. For example, a sterilized optical tag and cell culturing media were cultured for about twenty-four hours to ensure full internalization.

For example, in at least some example embodiments, a nanowire-carrying wafer may be immersed in ethanol and sonicated using an ultrasonic cleaner for about ten minutes so as to separate the nanowires from the wafer. After removing the wafer and centrifuging the remaining solution, the nanowires may be separated from the ethanol, re-dispersed in phosphate-buffered saline solution, and finally sterilized, under a UV lamp for about one hour in a laminar flow hood before culturing with the cells. To mimic the in vivo subretinal layer environment, the cells may be thawed on a layer of fibrin hydrogen gel. A fibrin hydrogel coated glass coverslip may be made by adding about 100 µl of a fibrin precursor solution (e.g., 5 mg/ml fibrinogen (Sigma Aldrich) and 10 units/ml thrombin (Sigma Aldrich) in 1X PBS) onto a 18 mm diameter glass coverslip and allowing crosslinking for about 30 minutes. The nanowire-carrying cells may then be seeded onto 5 mg/ml fibrin hydrogel coated coverslip and allowed to attach and spread over twenty-four hour period. Following this first predetermined period, a 150 µL solution of nanowire containing phosphate-buffered saline may be added and the cells allowed to phagocytose nanowires over another twenty-four hour period. Following this second predetermined period, the samples may be rinsed with media (e.g., three times) so as to remove free-floating nanowires.

After internalization, the target cells may be observed using a multi-modality imaging system, like the multi-modal imaging system 10 illustrated in FIG. 1. As demonstrated in FIG. 5A and FIG. 5B, the optical tags internalized by the target cells are able to provide contrast in both imaging modality. Four target cells are specifically identified 510, 520, 530, 540. From the distribution of the target cells 510, 520, 530, 540, it can be notice that the images from two imaging modality (i.e., FIG. 6A and FIG. 6B) matched well, confirming that enhanced contrast can be provided by the optical tags. FIG. 5C are lasing emission spectra for the target cells 510, 520, 530, 540. Such lasing emission spectra can be used to differentiate different cells in the same field of view for individual cell tracking.

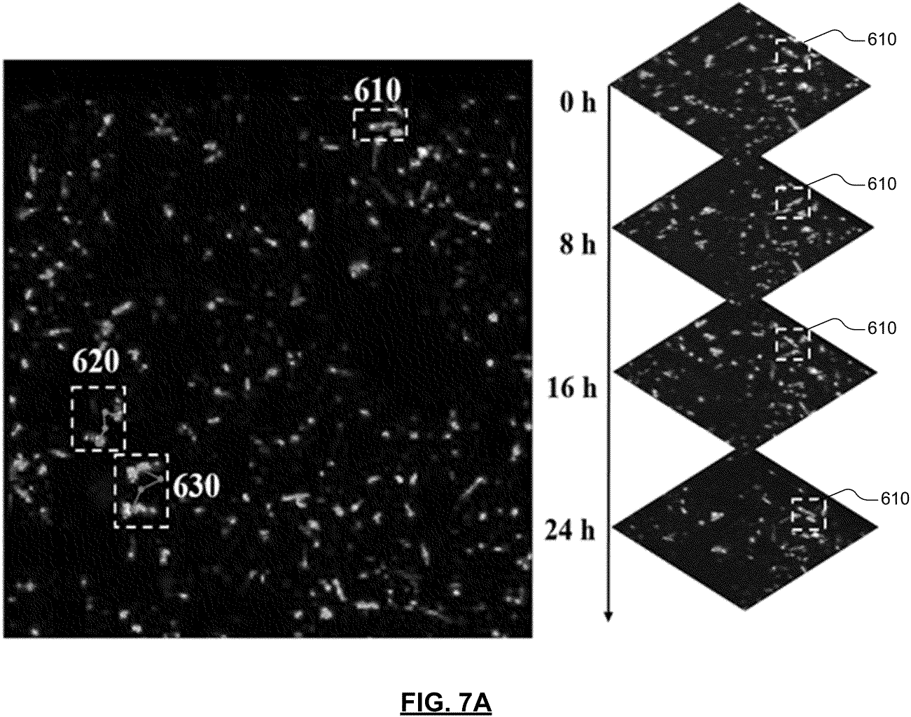

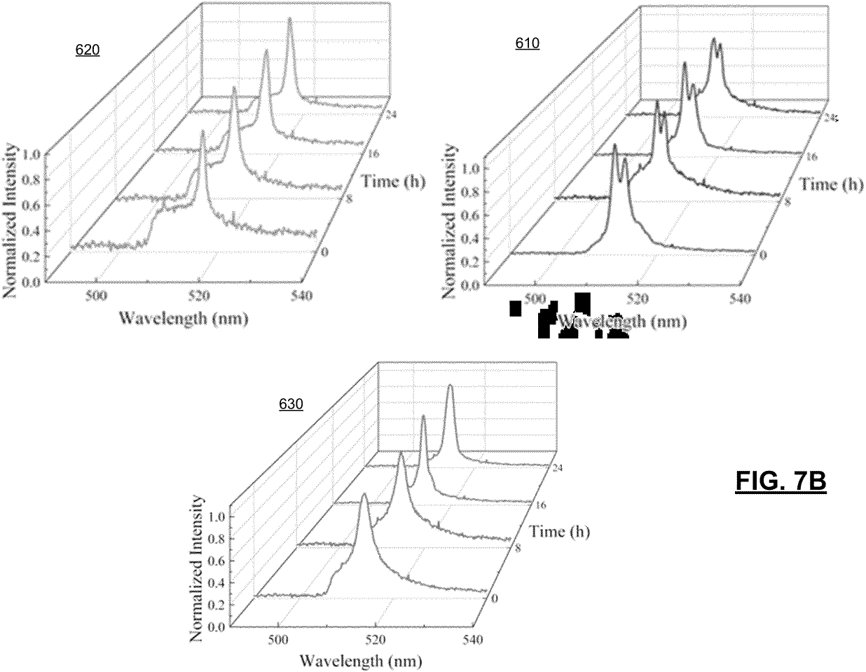

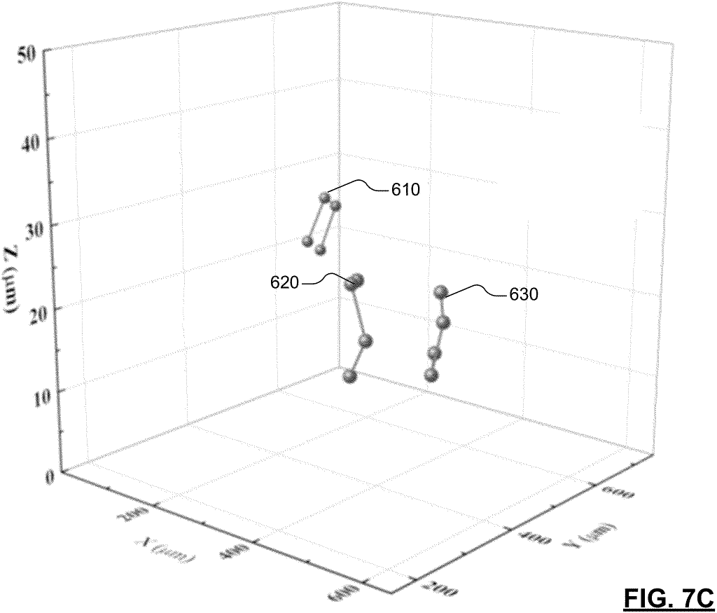

FIG. 7A is a fluorescence image of target cells (e.g., macrophages) labeled using optical tags (e.g., CdS nanowires) and cultured on a layer of scattering hydrogen gel. For example, a sterilized optical tag and cell culturing media were cultured for about twenty-four hours to ensure full internalization. After internalization, the target cells were observed using a multi-modality imaging system, like multi-modal imaging system 10 illustrated in FIG. 1. FIG. 7A illustrates the overlapping trajectories of migration of three selected cells 610, 620, 630 extracted from the fluorescence microscopy images over time. The right inset in FIG. 7A illustrates migration of the example target cell 610 over time through multiple frames. For example, the target cells 610, 620, 630 were observed successively, for example every eight hours during twenty-four hours period. A marker on the petri dish may be exploited to retrieve the field of view for each observation. The identities of tracked macrophages may be verified using lasing emission spectra of the optical tags. For example, FIG. 7B, are the lasing emission spectra collected from the target cells 610, 620, 630. During each observation, it is clear that the lasing emission spectra from the respective target cell 610, 620, 630 were unchanged and different from the lasing spectra of other target cells 610, 620, 630. Therefore, based on the identification information provided from FIG. 7B, the individual cell migration trajectories can be extracted from OCT images, as illustrated in FIG. 7C.

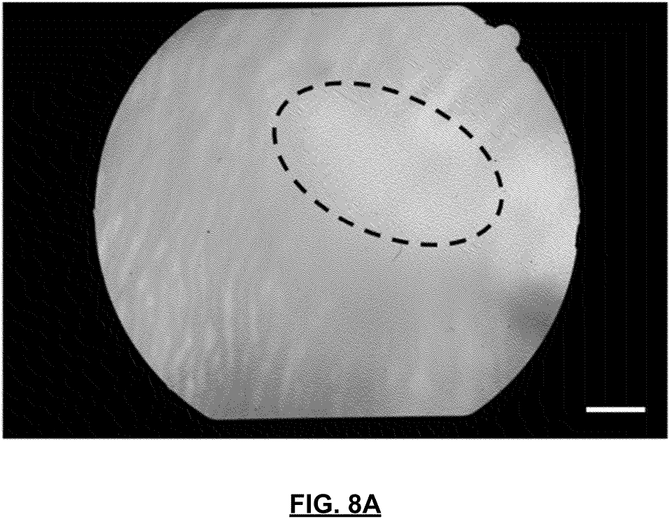

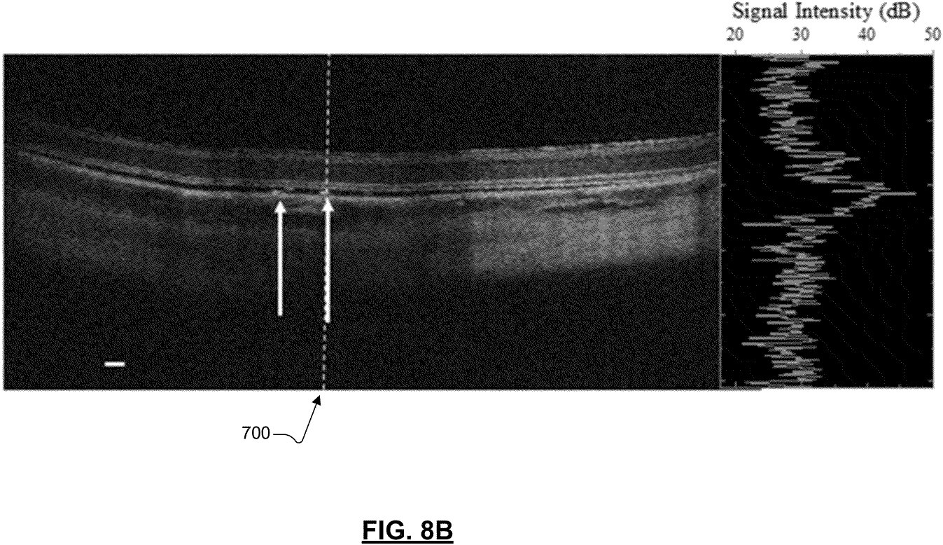

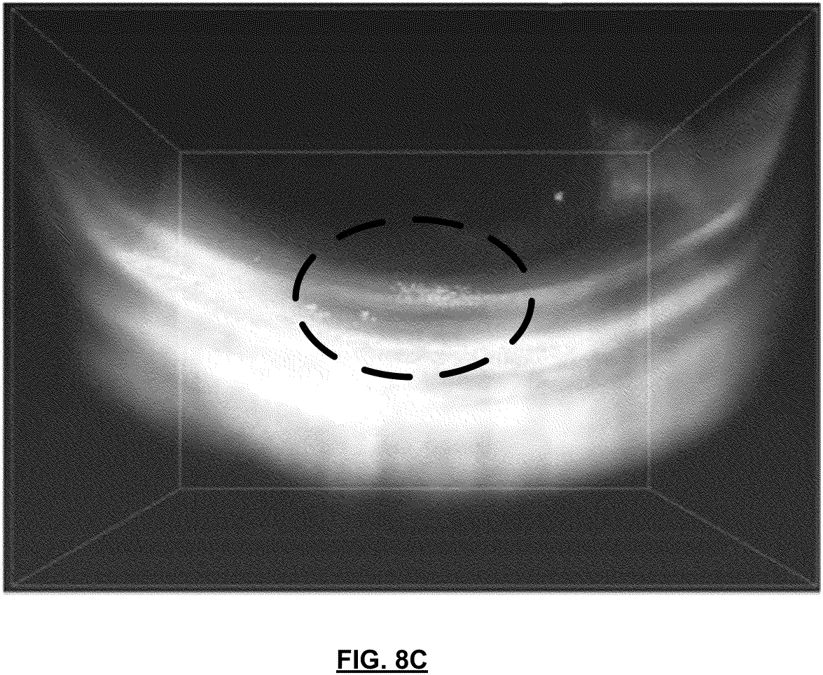

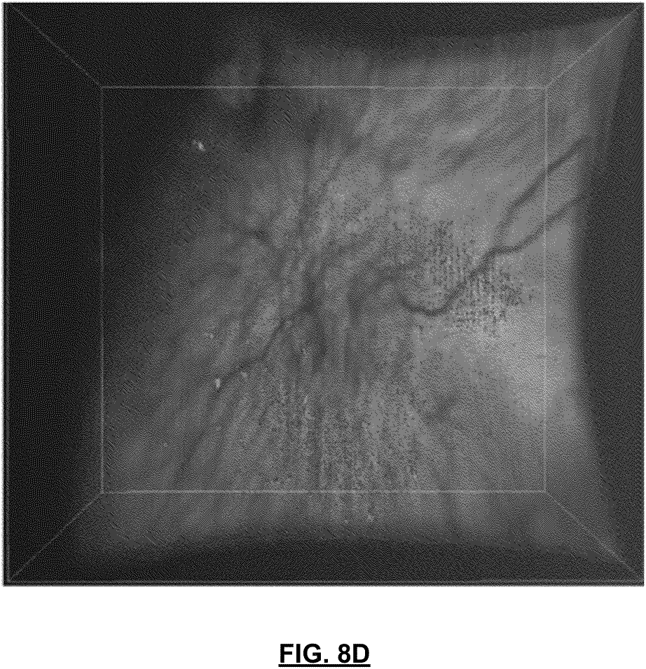

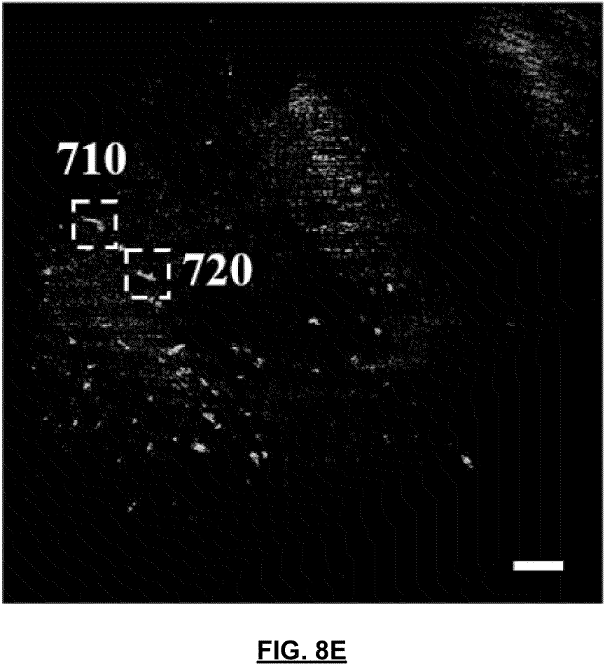

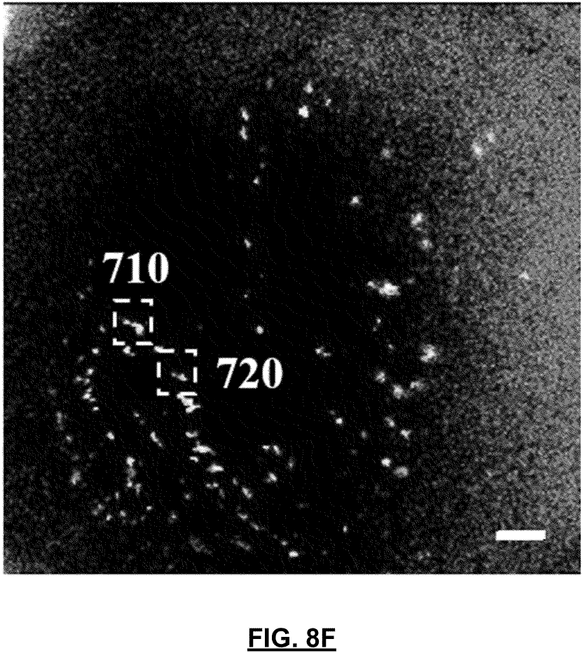

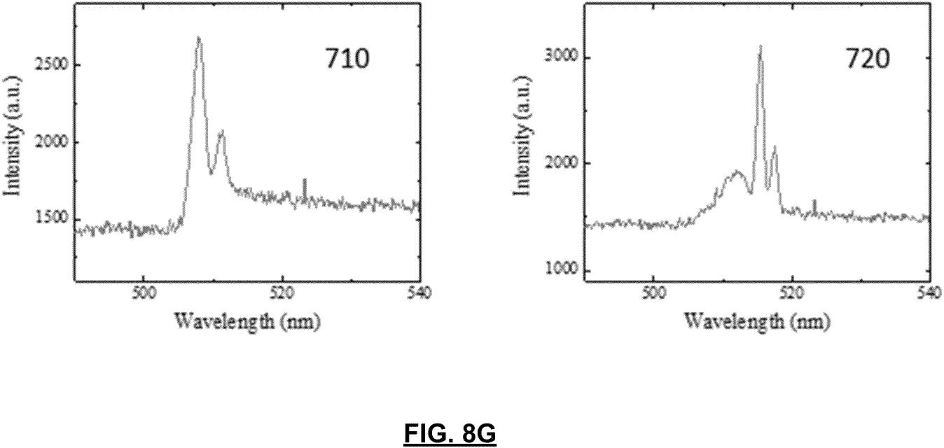

A sterilized optical tag and cell culturing media were cultured for about twenty-four hours to ensure full internalization. After internalization, a cell-enriched solution was prepared. The cell-enriched solution may be prepared, for example, using a centrifuge process. The cell-enriched solution (e.g., about 20 µL) may be injected into a sub-retinal layer, as shown in FIG. 8A, using a blunt-gouge needle. The region of the sub-retinal injection is circled in FIG. 8A.

In at least some example embodiments, an OCT system may be used for real-time guidance and assisting in confirming the injection depth by providing the anatomy of the injected tissue (e.g., retinal layers) at about 10 frames per second. For example, FIG. 8B is a cross-sectional illustrated acquired by the OCT of the injection area. The arrows indicated the locations of the target cells including the optical tags. The right inset is a signal intensity curve collected along the dashed line 700. By analyzing the signal profile along the dashed line 700, one could notice that the nanowire lasers provided a significant 30 dB OCT signal enhancement compared to the surround retinal layers. Based on this signal enhancement, the three-dimensional distribution of the optical tags in the subretinal layer may be determined, for example, using thresholding segmentation.

FIGS. 7C and 7D show the side and top views of the three-dimensional OCT image reconstruction, respectively. For example, in FIG. 7C the retina structure is exhibited in white and the optical tag distribution is shown as a plurality of dots circled. From this image, it can be determined that most of the optical tags were attached under the retina layer and a portion of the optical tags were on the retina layer due to subretinal injection leakage.

FIG. 8E is an X-Y plane projection of the spatial distribution of the optical tags in the same field of view as FIG. 8C. FIG. 8F is a fluorescence microscopy image of the spatial distribution of the optical tags in the same field of view as FIG. 8C. As demonstrated, the distribution acquired from OCT matches the fluorescence image so as to verify the locations acquired by thresholding. To distinguish different cells, lasing emission from the optical tags needs to be collected. For example, in FIG. 8G, two selected lasing spectra were collected from the two locations 710, 720 isolated in FIGS. 8E and 8F, proving that the lasing spectra could be collected under the retina layer for living animals.

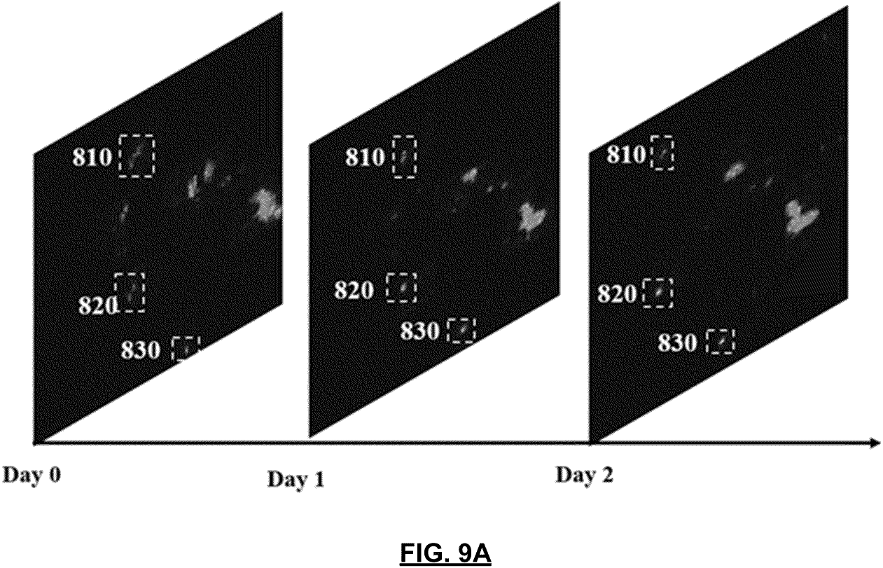

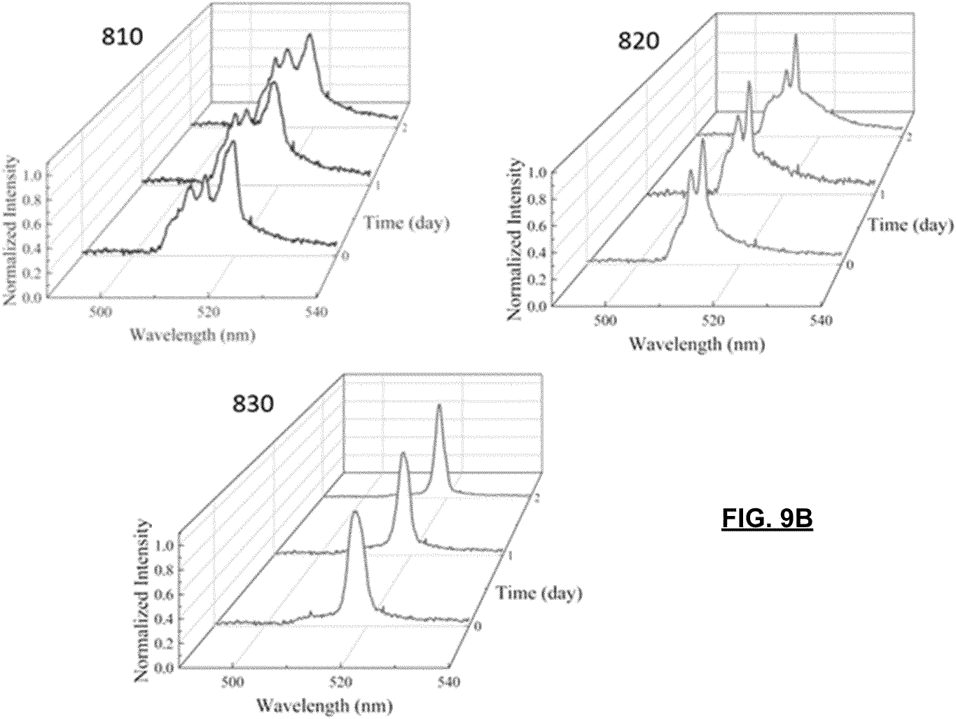

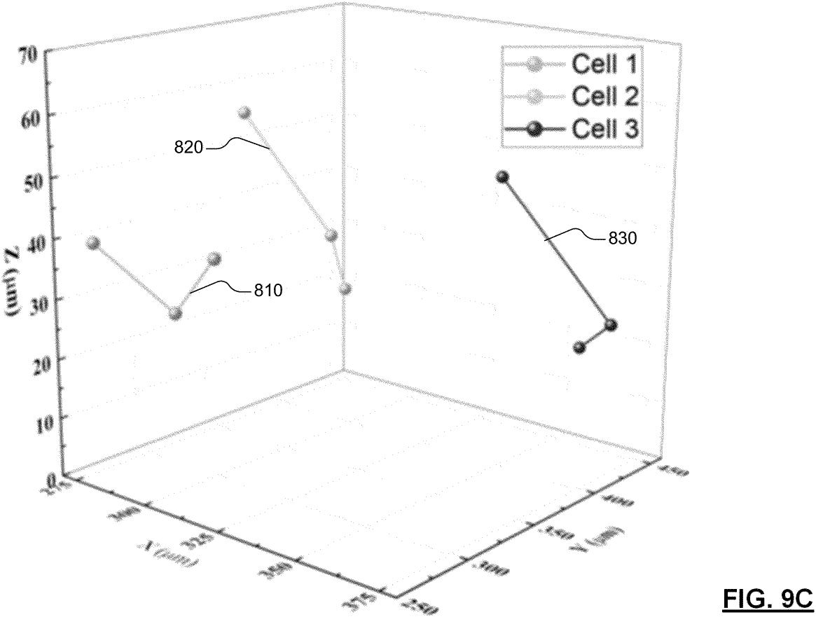

After the localized retinal detachment was recovered, the optical tag migration was observed successively for three days. In FIG. 9A, a series of fluorescence images of the interested area are shown. Three target cells are specifically tracked 810, 820, 830. During the observation, the lasing emission spectra remained unchanged and were utilized as identity verification for distinguishing different optical tags. For example, FIG. 9B illustrates the lasing emission spectra for the three selected cells. Because the identity of optical tags were verified, the individual macrophage migration trajectory can be extracted from the OCT result. The three-dimensional cell migration trajectories are illustrated in FIG. 9C. In this way, the feasibility of in vivo single-cell migration tracking with a multi-modality imaging system and optical tags is illustrated.

The techniques described herein or portions thereof may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An imaging system for cell-based therapies, the imagining system comprising:
one or more optical tags configured for insertion into a cell or biological tissue, wherein each of the one or more optical tags has a contrasting feature and includes a fluorescent material;
an excitation light source configured to illuminate the one or more optical tags;
a detector configured to measure optical emission of the one or more optical tags;
an imaging subsystem configured to determine a three-dimensional location of each of the one or more optical tags in the cell or biological tissue; and
a controller in electrical communication with the excitation light source, the detector, and the imaging subsystem.

2. The imaging system of claim 1, wherein the imaging subsystem is configured to determine the three-dimensional location of the one or more optical tags using optical coherence tomography (“OCT”).

3. The imaging system of claim 2, wherein each of the one or more optical tags exhibits a first refractive index that is different from a second refractive index of a surrounding medium of the cell or biological tissue, the first refractive index defining the contrasting feature.

4. The imaging system of claim 2, wherein the one or more optical tags includes a first optical tag and a second optical tag, the first optical tag exhibiting a first refractive index, the second optical tag exhibiting a second refractive index, and at least one of the first and second refractive indexes being different from a third refractive index exhibited by a surrounding medium of the cell or biological tissue or the first refractive index being different from the second refractive index, the first, second, and third refractive indexes defining the contrasting feature.

5. The imagining system of claim 2, wherein the one or more optical tags includes a first optical tag and a second optical tag, the first optical tag having a first shape, and the second optical tag having a second shape that is different from the first shape, the first and second shapes defining the contrasting feature.

6. The imagining system of claim 2, wherein the one or more optical tags includes a first optical tag and a second optical tag, the first optical tag having a first laser emission, and the second optical tag having a second laser emission that is different from the first laser emission, the first and second laser emissions defining the contrasting feature.

7. The imagining system of claim 2, wherein the one or more optical tags includes a first optical tag and a second optical tag, the first optical tag having a first color, and the second optical tag having a second color that is different from the first color, the first and second colors defining the contrasting feature.

8. The imagining system of claim 1, wherein at least one optical tag of the one or more optical tags has a core-shell structure including an air-containing core and a polymeric shell, the air-containing core and the polymeric shell defining the contrasting feature.

9. The imagining system of clam 8, wherein the imaging subsystem is configured to determine the three-dimensional location of the one or more optical tags using an ultrasound imagining subsystem.

10. The imagining system of claim 1, wherein the one or more optical tags includes a first optical tag and a second optical tag, the first optical tag exhibiting a first refractive index that is different from a second refractive index of a surrounding medium of the cell or biological tissue, and the second optical tag having a core-shell structure including an air-containing core and a polymeric shell, the first refractive index, the air-containing core, and the polymeric shell defining the contrasting feature.

11. The imaging system of claim 1, wherein the one or more optical tags are independently selected from a group consisting of: nanowires, semiconductor ring resonators, microspheres, photonic crystals, and combinations thereof.

12. The imagining system of claim 1, wherein at least one optical tag of the one or more optical tags is coated with a biocompatible coating.

13. The imaging system of claim 12, wherein the biocompatible coating includes one or more materials selected from the group consisting of: diallyl dimethylammonium chloride (PDDA), polyacrylic acid (PAA), collagen, polyethylene glycol (PEG), polydimethylsiloxane (PDMA), and combinations thereof.

14. The imaging system of claim 12, wherein the biocompatible coating includes a first coating and a second coating, the first coating surrounding the at least one optical tag of the one or more optical tags, and the second coating surrounding the first coating,
wherein the first and second coatings each include one or more materials independently selected from the group consisting of: diallyl dimethylammonium chloride (PDDA), polyacrylic acid (PAA), collagen, polyethylene glycol (PEG), polydimethylsiloxane (PDMA), and combinations thereof.

15. The imaging system of claim 1, wherein each of the one or more optical tags has a largest dimension less than about 20 µm.

16. The imaging system of claim 1, wherein each of the one or more optical tags has an length greater than or equal to about 3 µm to less than or equal to about 10 µm, and an average diameter of about 200 nm.

17. The imaging system of claim 1, wherein the excitation light source is a pulsed laser.

18. The imaging system of claim 1, wherein the detector is at least one of a photodiode or a spectrometer.

19. An imaging system for cell-based therapies, the imagining system comprising:
one or more optical tags configured for insertion into a cell or biological tissue, wherein each of the one or more optical tags has a contrasting feature and includes a fluorescent material, wherein the contrasting feature is defined by at least one of a refractive index, shape, color, and laser emission of each optical tag of the one or more optical tags;
an excitation light source configured to illuminate the one or more optical tags;
a detector configured to measure optical emission of the one or more optical tags;
an optical coherence tomography (“OCT”) imaging subsystem configured to determine a three-dimensional location of each of the one or more optical tags in the cell or biological tissue; and
a controller in electrical communication with the excitation light source, the detector, and the imaging subsystem.

20. The imaging system of claim 19, wherein at least one optical tag of the one or more optical tags has a core-shell structure including an air-containing core and a polymeric shell, the air-containing core and the polymeric shell defining another contrasting feature.
